(19) United States
(12) Patent Application Publication
(10) Pub. No.: US 2018/0207390 A1
(43) Pub. Date: Jul. 26, 2018

(54) INHALER DEVICE FOR INHALABLE LIQUIDS

(71) Applicant: Medical Developments International Limited, Scoresby (AU)

(72) Inventors: Greg Rowland, Scoresby (AU); Glenn Gilbert, Scoresby (AU); Edward Linacre, Abbotsford (AU); Viktor Legin, Abbotsford (AU)

(21) Appl. No.: 15/745,879

(22) PCT Filed: Jul. 19, 2016
(86) PCT No.: PCT/AU2016/050639

§ 371 Date: Jan. 18, 2018

(30) Foreign Application Priority Data
Jul. 20, 2015 (AU) 2015902864
Jul. 20, 2015 (AU) 2015902868
Jul. 20, 2015 (AU) 2015902881

(51) Int. Cl. A61M 16/14; A61M 15/06; A61M 15/00; A61M 16/20

(52) U.S. Cl. CPC A61M 16/142; A61M 15/06; A61M 15/0043; A61M 2209/06; A61M 16/20; A61M 2207/00; A61M 15/0035

(57) ABSTRACT

The present invention provides a new inhaler device for the storage and administration of inhalable liquids to a patient offering one or more advantages or improvements over known inhalers, particularly inhalers for the delivery of halogenated volatile liquids such as methoxyflurane for use as an analgesic.

### FIELD

The present invention relates to an inhaler device for inhalable liquids, in particular for the storage and/or administration of inhalable volatile liquids such as halogenated volatile liquids, to a patient.

### BACKGROUND

The storage and administration of inhalable liquids to patients that comprise active agents, or that are themselves the active agent, commonly presents challenges. Due to patient preference and ease of self-administration or administration in a hospital setting or other settings as required, active agents such as therapeutic agents or pharmaceutical agents, are often formulated for oral delivery in the form of tablets and capsules, nasal delivery in the form of sprays and liquid formulations for intravenous delivery.

Where it is advantageous to administer active agents to a patient's lungs, for example to treat or alleviate respiratory diseases, the active agent may be administered by the oral inhalation route, alone or in combination with the intranasal route. Suitable inhaler devices may include, for example, metered dose inhalers and dry powder inhalers. These types of oral inhalation devices typically require pressurised means to deliver the active agent to the desired site of action in the lungs. In addition, liquids that contain active agents or that are themselves the active agent usually require transformation into an inhalable, respirational, form at the point of administration to be suitable for delivery by the inhalation route.

Transforming a liquid into an inhalable form, such as by nebulisation or aerosolizing into respirational sized droplets or heating to form a vapour, requires delivery devices to include moving, mechanical, heating and/or electrical means which adds to the complexity of the design, manufacturing and end user costs, operability and/or patient use.

The use of volatile liquids as active agents or comprising active agents is known. One such example is halogenated volatile liquids. Halogenated volatile liquids have been described as useful for inducing and/or maintaining anaesthesia (including amnesia, muscle paralysis, and/or sedation) and/or analgesia and may therefore be useful as anaesthetics and/or analgesics. The anaesthetic properties of fluorinated compounds have been known since at least 1946 (Robbins, B. H. J Pharmacol Exp Ther (1946) 86: 197-204). This was followed by the introduction of fluoroxene, halothane and methoxyflurane into clinical use in the 1950s and the subsequent development of enflurane, isoflurane, sevoflurane and desflurane which are in clinical use in some countries today (Terrell, R. C. Anesthesiology (2008) 108 (3): 531-3).

Halogenated volatile liquids, when used for general anaesthesia, may be delivered to a patient under positive pressure via a delivery system that includes a vaporizer and a flow of breathable carrier gas. More recently, halogenated volatile liquids have been formulated for use in local or regional anaesthesia and delivery via non-inhalation routes. Examples include formulation as: microdroplets for intradermal or intravenous injection (e.g. U.S. Pat. No. 4,725,442); aqueous solutions for intrathecal or epidural delivery (e.g. WO2008/036858); swab, droplets, spray or aerosol for transmucosal delivery (e.g. WO2010/025505); aqueous based solutions comprising an extractive solvent in an amount effective to reduce the volatility, vaporisation or evaporation of the volatile anaesthetic for transdermal, topical, mucosal, buccal, rectal, vaginal, intramuscular, subcutaneous, perineural infiltration, intrathecal or epidural delivery (e.g. WO2009/094460, WO2009/094459); compositions suitable for formulation into a medical patch (e.g. WO2014/143964); compositions suitable for formulation as a solution, suspension, cream, paste, oil, lotion, gel, foam, hydrogel, ointment, liposome, emulsion, liquid crystal emulsion and nanoemulsions for topical, intrathecal, epidural, transdermal, topical, oral, intra-articular, mucosal, buccal, rectal, vaginal, intramuscular, intravesical and subcutaneous delivery (e.g. WO2008/070490, WO2009/094460, WO2010/129686); and stable and injectable liquid formulations (WO2013/016511).

The main consideration(s) for the safe storage and handling of volatile liquids commonly include vapour pressure build up, the robustness of the container and the integrity of the container seal(s). The chemical nature of the volatile liquid may also be important if the active agent is capable of permeating, solubilizing or otherwise reacting with the container material(s) upon storage. A number of storage containers for halogenated volatile liquids have been described including: rigid polymeric containers as a replacement for glass vials, such as capped bottles large tanks, shipping containers (e.g. WO1999/034762, WO2012/116187); rigid polymeric bottles fitted with a gasketless valve assembly and pliable containers with a threaded spout for fluid connection to deliver liquid anaesthetics to an anaesthetic machine or vaporizer (e.g. WO2010/135436, WO2013/106608, WO2013/149263, WO2015/034978); a container with a capped membrane for delivering a stored liquid anaesthetic to a vaporizer via a slotted tube (WO2009/117529); and rigid polymeric and aluminium containers optionally coated with materials to impart or enhance vapour barrier characteristics or container inertness (e.g. WO2002/022195, WO2003/032890, WO2010/129796).

Despite the various advances in formulating volatile liquids in non-inhalable forms, such as the halogenated volatile liquids, as well as containers to store them, there still remains a need for inhalable forms of volatile liquids and devices to store and/or administer them to patients.

Attempts to design new inhalers for inhalable medicines in general are ongoing. For example, WO2008/040062 describes a diverse number of inhaler device concepts that depend on complex constructions and moving parts for storing and/or delivering inhalable liquids and powdered solids into a user's mouth or nose. The various devices described are adapted to hold one or two medicament containers in the form of pressurised canisters, ampoules, vials and plungers. The devices are described as being activated by sliding an outer wall of the device in relation to an inner wall of the device to deliver the liquid medication from a medication container. In a number of embodiments, the device includes a moveable mouthpiece which deploys in order to open the air pathway. The device is also described as including one or more one-way valves to provide a unidirectional air flow for one or both inhaled air and exhaled air (a series of one-way valves to direct the flow of inhaled and exhaled air has also been generally described in WO2007/033400 which is an incorporation by reference of the device described in WO1997/003711).

When required for use, the devices of WO2008/040062 are claimed as being capable of releasing the medication by punching means namely two punches to perforate the two frangible ends respectively of a medication container having frangible ends, although various other means are generally described including: pressurised means (e.g. by a pressurised canister); frangible means (e.g. by rupturing an ampoule with a striker or by punching a frangible membrane or seal of a vial with punch means); crushable means (e.g. by crushing a vial with a plunger); dislodging means (e.g. by dislodging an unscrewed cap from a vial); and plunging means (e.g. by plunging the medication from the plunger barrel).

However, inhalable liquids such as halogenated volatile liquids require an effective air chamber into which the vapour may evaporate and allow an effective airflow through the air/vapour chamber for delivery to a patient. Accordingly, embodiments such as those described in, for example, FIGS. 48A, 48B, 48C, 49A, 49B, 50A, 50B, 51A, 51B, 56A, 56B, 57, 58A, 58B, 58C and 58D of WO2008/040062, would not be expected to work in practice as the evaporative means (or wick) is prevented from being effectively exposed to the released liquid by the walls of the liquid storage container itself.

The present invention provides a new inhaler device for the storage and administration of inhalable liquids to a patient offering one or more advantages or improvements over known inhalers, particularly inhalers for the delivery of halogenated volatile liquids such as methoxyflurane for use as an analgesic. The device is capable of storing and administering an inhalable liquid by providing an internally stored sachet comprising a passive evaporation support material pre-loaded with the liquid. The present invention also provides a new and inventive container adapted for use in an inhaler device in the form of a storage sachet comprising a passive evaporation support material pre-loaded with an inhalable liquid such as a halogenated volatile liquid. The storage sachet is adapted for storing the inhalable liquid within the device and releasing the stored liquid from the passive evaporation support material in the form of a vapour for inhalation by a patient when opened. The device comprising the storage sachet offers an easy to use, pre-loaded (i.e. primed for use), readily portable and low-cost manufactured device and storage container which may also provide further reductions in shipping, storage and disposal costs.

### SUMMARY

According to a first aspect of the invention there is provided an inhaler device for the delivery of an inhalable liquid to a patient, said device comprising:
(1) An inhaler body;
(2) At least one air inlet opening;
(3) At least one vapour inhalation opening; and
(4) A storage sachet comprising a vapour impermeable film or foil for sealingly storing a passive evaporation support material pre-loaded with the inhalable liquid;
   wherein the inhalable liquid is released from the storage sachet into the inhaler body in the form of a vapour for administration to a patient when the sachet is opened and further wherein the air inlet opening and vapour inhalation opening provide an air/vapour pathway through the inhaler body.

According to an alternative aspect of the invention there is provided an inhaler device for the delivery of an inhalable liquid to a patient, said device comprising:
(1) An inhaler body comprising a passive evaporation support material for receiving the inhalable liquid;
(2) At least one air inlet opening;
(3) At least one vapour inhalation opening; and
(4) A storage sachet comprising a vapour impermeable film or foil for sealingly storing the inhalable liquid;
   wherein the stored liquid is released from the storage sachet into the inhaler body and onto the passive evaporation support material to form a vapour for administration to a patient when the sachet is opened and further wherein the air inlet opening and vapour inhalation opening provide an air/vapour pathway through the inhaler body.

According to a second aspect there is provided a storage sachet for use in an inhaler device wherein the storage sachet comprises a vapour impermeable film or foil for sealingly storing an inhalable liquid, preferably a passive evaporation support material pre-loaded with an inhalable liquid, particularly a halogenated volatile liquid, and for releasing the liquid for delivery to a patient as a vapour upon opening. In one embodiment, the storage sachet is entirely formed from a vapour impermeable film or foil adapted to sealing store the inhalable liquid, preferably a passive evaporation support material pre-loaded with a halogenated volatile liquid. In another embodiment, the storage sachet is partially formed from a vapour impermeable film or foil and partially formed from a rigid or semi-rigid base portion wherein the vapour impermeable film or foil is adapted to sealing store the inhalable liquid, preferably a passive evaporation support material pre-loaded with a halogenated volatile liquid.

In one embodiment according to the first, alternative and second aspects of the invention, the inhalable liquid is a halogenated volatile liquid. In a further embodiment the halogenated volatile liquid is selected from the group consisting of halothane (2-bromo-2-chloro-1,1,1-trifluoroethane), sevoflurane (fluoromethyl-2,2,2-trifluoro-1-(trifluroromethyl)ethyl ether), desflurane (2-difluoromethyl-1,2,2,2-tetrafluoroethrylether), isoflurane (1-chloro-2,2,2-trifluoroethyldifluoromethyl ether), enflurane (2-chloro-1,1,2-trifluoroethyldifluoromethyl ether) and methoxyflurane (2,2-dichloro-1,1-difluoroethylmethyl ether). In a preferred embodiment, the inhalable liquid is methoxyflurane for use as an analgesic.

### BRIEF DESCRIPTION OF THE FIGURES

FIG. 1 shows a prior art inhaler device, referred to as the Green Whistle™ inhaler device (Medical Developments International Limited) that is currently used to administer methoxyflurane.

FIG. 2 shows an inhaler device according to an embodiment of the invention comprising a storage sachet with a pull tab (FIG. 2A) for opening by the user (FIG. 2B) to release the inhalable liquid vapour into the air intake chamber for inhalation by the patient (FIG. 2C).

FIG. 3 shows a storage sachet according to an embodiment of the invention comprising a planar base portion (FIG. 3A) and a pull tab for opening by the user (FIG. 3B) to remove the vapour impermeable film thereby exposing the passive evaporation support material to allow release of the pre-loaded inhalable liquid as a vapour from its surface (FIG. 3C).

FIG. 4 shows a storage sachet fastened to an inhaler body according to an embodiment of the invention.

FIG. 5 shows an inhaler device according to an embodiment of the invention comprising a storage sachet (FIG. 5A). A cross-sectional view A-A of the device of FIG. 5A is also presented to better illustrate the inhaler body of the device which comprises an air intake chamber, an air exit chamber comprising an air filtering means, a mouthpiece chamber and an internal shelf to partially divide the inhaler body along its longitudinal axis and terminating at the mouthpiece chamber to form the floor of the air intake chamber and the roof of the air exit chamber and further showing the storage sachet positioned within the air intake chamber and on the internal shelf (FIG. 5B).

FIG. 6 shows an exploded view of the inhaler device of FIG. 5 to better illustrate its components (FIG. 6A) and its assembly (FIG. 6B).

FIG. 7 shows a perspective view of an inhaler device having a puck-shaped body in accordance with an embodiment of the invention (FIG. 7A), and an exploded view (FIG. 7B) of the same device to better illustrate some internal components.

FIG. 8 shows an inhaler device according to an embodiment of the invention comprising a storage sachet located within the device and positioned above the passive evaporative support material (FIG. 8A) to release the inhalable liquid onto the passive evaporation support material when opened by ripping, unpeeling, pulling or similar (FIG. 8B).

FIG. 9 shows the comparative concentrations of methoxyflurane delivered by a device (FIG. 7) according to an embodiment of the invention and the prior art Green Whistle inhaler.

### DETAILED DESCRIPTION

Inhaler devices that are useful for administering inhalable liquids may be generally considered to operate by either passive or active means in order to deliver the active agent(s) to a patient. Inhaler devices with active means may include pressurized, moving, mechanical, heating and/or electrical means to, for example, nebulise, vaporize and/or generally deliver the active agent(s). In contrast, inhaler devices with passive means rely solely on the vaporisation or evaporation of the active agent(s) at ambient conditions and respiration of the patient to deliver the active agent(s).

The Analgizer™ inhaler device (Abbott Laboratories Corporation) is an example of a device that operates by passive means to deliver an inhalable liquid. According to the USPTO TESS database, the Analgizer™ was a registered, now lapsed, trademark in respect of an inhaler for the supervised self-administration of inhalation anaesthesia and was first used in 1968. The Analgizer™ was a very simple device that consisted of a white cylindrical polyethylene open-ended tube having a mouthpiece and an absorbent wick of polypropylene which was tightly rolled into a ‘Swiss-roll’ shape, i.e. cross-sectional view. The inhalation anaesthetic, methoxyflurane (15 mL), was poured into the open ended base of the inhaler and onto the tightly wound wick, just prior to use. A patient was then able to self-administer the liquid anaesthetic by inhaling through the mouthpiece.

The Green Whistle™ inhaler device (Medical Developments International Limited) was subsequently developed during the 1990s and has since been used in Australia for the delivery of Penthrox®/™ (methoxyflurane) as an analgesic (1.5 mL or 3 mL, storage brown glass vial container with screw cap). Although similar in its simplicity of design to the Analgizer™, the Green Whistle™ device includes certain functional improvements such as the inclusion of a one-way valve at the base end to prevent drug vapour loss from the device upon patient exhalation and an activated carbon (‘AC’) chamber designed to be externally fit into a dilution hole in the mouth piece to filter exhaled drug vapours. Additional design modifications to the base end included the introduction of cap lugs to assist removal of the cap from the glass vial used to store the drug dose to be delivered, a dome to facilitate the spread of the poured liquid onto the ‘S-shaped’ wick circumference (i.e. cross-sectional view) or, in the alternative to a dome, an inlet nipple to allow for the attachment of a breathable gas line to direct the gas through the device. The Green Whistle™ device is designed for single patient use.

Methoxyflurane (Penthrox®/™, Medical Developments International Limited) offers a non-narcotic, i.e. non-opioid analgesic alternative to common analgesics such as morphine and fentanyl. Methoxyflurane also presents an alternative to analgesics which are administered in oral tablet form or intraveneously to a patient and may therefore be particularly useful when rapid pain relief is required in clinical, surgical (e.g. pre- and post-operative) and/or emergency settings (e.g. emergency department and triage management as well as by first-responders such as paramedics and search and rescue teams). However, the Green Whistle™ device is currently the only device that is commercially available to administer methoxyflurane. According to the device's instructions for use, the administrator is required to hold the methoxyflurane bottle upright to use the base of the inhaler to loosen the bottle cap and then to remove the cap by hand before tilting the inhaler to a 45° angle and pouring the contents of the bottle into the base while rotating the device. An AC-chamber may be optionally fitted externally to the device either beforehand or afterwards. While the device is effective, the number of steps and separate components may present handling difficulties for the administrator or self-administrator, for example, in high-stress and/or emergency settings.

The present invention provides a new inhaler device and container for the storage and administration of inhalable liquids to a patient, such as halogenated volatile liquids, particularly methoxyflurane for use as an analgesic, the device and container having one or more advantages or improvements over known inhalers.

### Definitions

Unless otherwise herein defined, the following terms will be understood to have the general meanings which follow.

‘Active agent’ refers to therapeutic agents and non-therapeutic agents and compounds, formulations and compositions comprising them.

‘Alleviate’, ‘Alleviation’ and variations thereof refers to relieving, lessening, reducing, ameliorating or an improvement in the symptom(s) and/or underlying cause(s) of a condition and/or disease in a patient.

‘Delivery dose’ refers to the dose of inhalable liquid or active agent for administration to a patient.

‘Filter’, ‘Filtering’ and variations thereof refers to the ability of a substance to absorb, adsorb, capture, trap, scavenge, scrub or partially or entirely remove the inhalable volatile liquid vapour from the exhaled breath of a patient upon exhalation.

‘Halogenated volatile liquids’ refers to volatile liquids which (i) comprise at least one halogen atom selected from the group consisting of a chlorine (CI), bromine (Br), fluorine (F) and iodine (I) atoms, or (ii) comprise an active agent which comprises at least one halogen atom selected from the group consisting of a chlorine (CI), bromine (Br), fluorine (F) and iodine (I) atoms. In some embodiments, halogenated, particularly fluorinated, hydrocarbons and halogenated, particularly fluorinated, ethers may be preferred. In some embodiments, halogenated ethers may be particularly preferred and include but are not limited to, halothane (2-bromo-2-chloro-1,1,1-trifluoroethane), sevoflurane (fluoromethyl-2,2,2-trifluoro-1-(trifluroromethyl)ethyl ether), desflurane (2-difluoromethyl-1,2,2,2-tetrafluoroethrylether), isoflurane (1-chloro-2,2,2-trifluoroethyldifluoromethyl ether), enflurane (2-chloro-1,1,2-trifluoroethyldifluoromethyl ether) and methoxyflurane (2,2-dichloro-1,1-difluoroethylmethyl ether).

‘Inhalable liquid’ refers to liquids that comprise active agents or that are themselves the active agent and that are readily inhalable or capable of being or adapted to be inhaled by a patient. In some embodiments, inhalable volatile liquids, particularly halogenated volatile liquids are preferred.

‘Inhalation’, ‘Inhalable’ and variations thereof refers to the intake of, for example but not limited to air, breathable gases, inhalable liquids, by a patient and includes both oral and nasal inhalation. In some embodiments, oral inhalation is particularly preferred.

‘Patient’ refers to both human and veterinary patients. In some embodiments, human patients may be particularly preferred. Reference to a patient will therefore be understood to mean the person or animal to whom the inhalable liquid is administered to and in the case of human patients, will be understood to include administration by self-administration.

‘Pharmaceutical agent’ refers to a drug, or a compound, formulation or composition that comprises a drug, for the treatment of symptom(s) and/or underlying cause(s) of a condition and/or disease in a patient. The term pharmaceutical agent may be used interchangeably with therapeutic agent or active agent.

‘Respiratory’, ‘Respirational’ and variations thereof refers to the act of respiring, breathing, inhaling and exhaling, such as for example but not limited to air, breathable gases, inhalable liquids and active ingredients, by a patient.

‘Room temperature’ refers to ambient temperatures which may be, for example, between 10° C. to 40° C. but more typically between 15° C. to 30° C.

‘Therapeutic agent’ refers to an active agent, or a compound, formulation or composition (including biological compounds, formulations and compositions) that comprises an active agent, that is capable of treating a patient or offers a therapeutic or medical benefit to a patient or that has or that requires regulatory and/or marketing approval for therapeutic use in a patient. Therapeutic agents include pharmaceutical agents. In contrast, a ‘Non-therapeutic agent’ will be understood to mean an active agent which may not have or require regulatory and/or marketing approval for a therapeutic use such as, for example, smokeless tobacco products and electronic cigarettes, or does not have a recognised or identified therapeutic use but may be used by a patient for a non-therapeutic reason such as general health, wellbeing or physiological benefit such as, for example, nutraceutical products.

‘Treat’, ‘Treatment’ and variations thereof refers to the alleviation, modulation, regulation or halting of the symptom(s) and/or underlying cause(s) of a condition and/or disease in a patient. In some embodiments treatment may include preventative or prophylactic treatment.

‘Volatile liquids’ refers to substances that predominantly exist in a liquid form but readily form vapours, evaporate or vaporize such that they partially exist in a vapour form under ambient conditions for example, at room temperature and at normal atmospheric pressures.

### EMBODIMENTS

Embodiments will now be described with reference to the non-limiting examples.

The present device comprises a passive evaporation support material pre-loaded with the inhalable liquid to provide a portable, ready-to-use, all-in-one, drug storage and delivery device. In comparison to the prior inhaler devices for methoxyflurane, the present device provides easy administration, in particular self-administration when rapid pain relief is required, for example, in emergency, non-hospital, isolated, outdoor environment, sporting, humanitarian aid and/or field operation environments.

According to the first aspect there is provided an inhaler device for the delivery of an inhalable liquid to a patient, said device comprising:
(1) An inhaler body;
(2) At least one air inlet opening;
(3) At least one vapour inhalation opening; and
(4) A storage sachet comprising a vapour impermeable film or foil for sealingly storing a passive evaporation support material pre-loaded with the inhalable liquid;
   wherein the stored liquid is released from the storage sachet into the inhaler body in the form of a vapour for administration to a patient when the sachet is opened and further wherein the air inlet opening and vapour inhalation opening provide an air/vapour pathway through the inhaler body.

According to an alternative aspect there is provided an inhaler device for the delivery of an inhalable liquid to a patient, said device comprising:
(1) An inhaler body comprising a passive evaporation support material for receiving the inhalable liquid;
(2) At least one air inlet opening;
(3) At least one vapour inhalation opening; and
(4) A storage sachet comprising a vapour impermeable film or foil for sealingly storing the inhalable liquid;
   wherein the stored liquid is released from the storage sachet into the inhaler body and onto the passive evaporation support material to form a vapour for administration to a patient when the sachet is opened and further wherein the air inlet opening and vapour inhalation opening provide an air/vapour pathway through the inhaler body.

In one embodiment the inhaler body comprises an air intake chamber comprising the air inlet opening and a mouthpiece comprising the vapour inhalation opening. The storage sachet will typically be located within the air intake chamber such that upon activation of the device by opening the storage sachet, the vapour will be released from the pre-loaded passive evaporation support means and into the air intake chamber where it is available to the patient for delivery upon inhalation through the mouthpiece.

The inhaler body is adapted to fasten the storage sachet in place. For example, the storage sachet may be fastened in place by sandwiching between an upper portion and a lower portion of the inhaler body which may optionally comprises fastening means or may be fastened by fastening means to an internal rim, shelf or floor of the inhaler body. In one embodiment the inhaler body optionally comprises a fastening portion selected from the group consisting of an upper and lower inhaler body portion, an internal rim, a shelf and a floor. Suitable fastening means may include, for example, adhesives, welds, screws, pins, hooks, rivets, snap-fit joint arrangements and male-female attachment arrangements. Accordingly, in one embodiment the inhaler body is adapted to fasten the storage sachet in place by sandwiching between an upper portion and lower portion of the inhaler body or by fastening to an internal rim, shelf or floor of the inhaler body. In a further embodiment the inhaler body fastens the storage sachet by an outer perimeter portion of the storage sachet.

According to the second aspect there is provided a storage sachet for use in an inhaler device as described herein wherein the storage sachet comprises a vapour impermeable film or foil for sealingly storing an inhalable liquid, preferably a passive evaporation support material pre-loaded with an inhalable liquid more preferably a halogenated volatile liquid, and for releasing the liquid for delivery to a patient as a vapour upon opening.

The storage sachet may be entirely or partially formed from a vapour impermeable film or foil. Vapour impermeable films may be a single layer or a laminate film comprising at least one vapour impermeable layer. The storage sachet may be sealed by suitable sealing means, such as by welding, for example, thermal welding, ultrasonic welding or by adhesives including peelable adhesives.

Examples of vapour impermeable films include but are not limited to polymeric films, metal foils (such as, for example, aluminium, nickel and alloys thereof) and combinations, including co-extruded polymeric films and/or foils such as laminate films, thereof. In one embodiment the vapour impermeable film is a single layer selected from a polymeric film or a metal foil. In another embodiment the vapour impermeable film is a laminate film comprising two or more layers selected from a polymeric film, a metal foil and combinations, including co-extruded polymeric films and/or foils, thereof. The laminate film may comprise a weldable layer made from a suitable weldable foil or polymeric film such as, for example, LLDPE. A weldable layer may assist with sealing the layers of a laminate together and/or sealing a vapour impermeable film comprising a weldable layer to the device. Processes suitable for welding include thermal and ultrasonic welding. The laminate film may comprise an adhesive layer including a peelable adhesive layer.

In one embodiment the polymeric film has a MVTR of less than 100 g/m²/24 h, preferably less than 50 g/m²/24 h. In one embodiment the polymeric film comprises a polymer selected from the group consisting of a polyolefin, a polymeric phthalate, a fluorinated polymer, a polyester, a nylon, a polyvinyl, a polysulfone, a natural polymer and combinations, including co-extruded polymers thereof including biaxially orientated polymers such as, for example, biaxially orientated polypropylene (BOPP). In one embodiment the polymeric film comprises a polymer selected from the group consisting of PP, PE, LDPE, LLDPE, HDPE, BOPP, 4-methylpentene, polymethylpentene polycyclomethylpentene, PEN, PET, PETP, PEI, PBT, PTT, PCT, Kel-F, PTFE, cellulose acetate, POM, PETG, PCTG, PCTA, nylon, PVA, EVOH, starch, cellulose, proteins and combinations, including co-extruded polymers, thereof.

In one embodiment the vapour impermeable film comprises PET. In another embodiment the vapour impermeable film comprises PET and a metal foil layer, preferably an aluminium foil layer. In one embodiment the vapour impermeable film comprises metalised PET (Met PET).

In one embodiment the vapour impermeable film comprises a co-extruded polymer layer adhered to a metalised PET layer adhered to an externally peelable LLDPE layer. In a further embodiment the co-extruded polymer layer is a biaxially orientated polymer, preferably BOPP. In another embodiment the vapour impermeable film comprises a layer of BOPP adhered to a metalised PET layer adhered to an externally peelable LLDPE layer.

In one embodiment, the storage sachet is entirely formed from a vapour impermeable film adapted to sealingly store the inhalable liquid, preferably a passive evaporation support material pre-loaded with an inhalable liquid, more preferably a halogenated volatile liquid. When the storage sachet is entirely formed from a vapour impermeable film it may be sealed by sealing an outer perimeter portion of the vapour impermeable film to itself. In another embodiment, the storage sachet is formed from a vapour impermeable film having a base portion wherein the vapour impermeable film is adapted to sealingly store the inhalable liquid, preferably a passive evaporation support material pre-loaded with an inhalable liquid, more preferably a halogenated volatile liquid, together with the base portion and further wherein the base portion is rigid or semi-rigid.

In one embodiment the base portion is formed from a polymer as described herein. To reduce manufacturing costs the base portion and the inhaler body may be formed from the same polymer. The base portion will typically be planar but may optionally comprise a receptacle portion for receiving the passive evaporation support material pre-loaded with the halogenated volatile liquid. When the storage sachet is formed from a vapour impermeable film having a base portion it may be sealed by sealing a perimeter edge of the base portion with an outer perimeter portion of the vapour impermeable film. Where the base portion comprises a receptacle portion, the perimeter edge of the base portion may be a lip of the receptacle. Further, the receptacle portion may form part of the inhaler device itself as further described in embodiments herein.

In one embodiment the storage sachet comprises an outer perimeter portion for fastening to the inhaler body.

When the storage sachet is positioned within the inhaler device for administration of the halogenated volatile liquid to the patient, the storage sachet may be opened by removing the vapour impermeable film or a portion thereof by peeling, pulling, tearing, ripping, perforating, puncturing or piercing.

To assist opening the storage sachet by peeling, pulling, tearing or ripping, the storage sachet may optionally comprise a pull tab which may protrude through an opening in the inhaler body, such as an air inlet opening or air exit opening, whereby it can be gripped and pulled by the user. Accordingly, in one embodiment the storage sachet comprises a pull tab adapted to open the storage sachet by peeling, pulling, tearing or ripping the vapour impermeable film. The pull tab may be made from any suitable material capable of connecting to the vapour impermeable film and withstanding the pulling or peeling forces required to open the storage sachet. The pull tab may be integrally formed and connected to the vapour impermeable film and in one embodiment the pull tab is integrally formed from the vapour impermeable film. The pull tab may also be independently formed and connected to the vapour impermeable film and in one embodiment the pull tab is made from a different material to the vapour impermeable film.

To assist opening the storage sachet by perforating, puncturing or piercing, the sachet may engage with the inhaler body which may optionally comprise a perforating, puncturing or piercing means operable by movement of the device. For example, when the inhaler body comprises a rotatable lid as further described in embodiments herein, the lid may comprise serrated projections which perforate, puncture or pierce open the vapour impermeable film upon rotating the lid open. Accordingly, in one embodiment the inhaler body comprises serrated projections adapted to open the storage sachet by perforating, puncturing or piercing the vapour impermeable film.

The present device is considered to be particularly useful for storing and administering a halogenated volatile liquid, particularly methoxyflurane for use as an analgesic. Accordingly, in one embodiment the storage sachet comprises a halogenated volatile liquid, preferably a passive evaporation support material pre-loaded with a halogenated volatile liquid. In a further embodiment the halogenated volatile liquid is selected from the group consisting of halothane (2-bromo-2-chloro-1,1,1-trifluoroethane), sevoflurane (fluoromethyl-2,2,2-trifluoro-1-(trifluroromethyl)ethyl ether), desflurane (2-difluoromethyl-1,2,2,2-tetrafluoroethrylether), isoflurane (1-chloro-2,2,2-trifluoroethyldifluoromethyl ether), enflurane (2-chloro-1,1,2-trifluoroethyldifluoromethyl ether) and methoxyflurane (2,2-dichloro-1,1-difluoroethylmethyl ether). In a preferred embodiment, the inhalable liquid is methoxyflurane for use as an analgesic.

Suitable delivery doses of inhalable liquid for administration to a patient by the present device may be determined by reference to, for example, regulatory approved dosage amounts. Suitable delivery doses of methoxyflurane for use as an analgesic will typically be less than 15 mL and preferably less than 12 mL and selected from the group consisting of 0.5 mL, 1 mL, 1.5 mL, 2 mL, 2.5 mL, 3 mL, 3.5 mL, 4 mL, 4.5 mL, 5 mL, 5.5 mL, 6 mL, 6.5 mL, 7 mL, 7.5 mL, 8 mL, 8.5 mL, 9 mL, 9.5 mL, 10 mL, 10.5 mL, 11 mL, 11.5 mL and 12 mL. In one embodiment the delivery dose of methoxyflurane for administration by the present device is selected from the group consisting of 1.5 mL, 3 mL and 6 mL.

The present device comprises a passive evaporation support material pre-loaded with the inhalable liquid and stored within a storage sachet to provide a portable, ready-to-use, all-in-one, drug storage and delivery device. In comparison to the prior inhaler devices for methoxyflurane, the present device provides easy administration, in particular self-administration when rapid pain relief is required, for example, in emergency, non-hospital, isolated, outdoor environment, sporting, humanitarian aid and/or field operation environments.

In one embodiment the passive evaporation support material stored within the storage sachet is adapted to form a single longitudinal airflow/vapour pathway though the vapour chamber when the storage sachet is opened. In another embodiment, the passive evaporation support material is adapted to form at least two independent longitudinal airflow/vapour pathways though the vapour chamber when the storage sachet is opened. In yet another embodiment, the passive evaporation support material is adapted to form three or more independent longitudinal airflow/vapour pathways though the vapour chamber when the storage sachet is opened.

In one embodiment the passive evaporation support material stored within the storage sachet is adapted to provide a single longitudinal airflow/vapour pathway though the vapour chamber when the storage sachet is opened and in one embodiment is planar.

In another embodiment the passive evaporation support material is adapted to form at least two independent longitudinal airflow/vapour pathways, three or more independent longitudinal airflow/vapour pathways, through the vapour chamber when the storage sachet is opened. Numerous examples of cross-sectional shapes which are capable of forming at least two, three or more independent longitudinal airflow/vapour pathways may be envisaged, some of which follow. The two, three or more independent longitudinal airflow/vapour pathways may be formed by the passive evaporation support material adopting a cross-sectional shape selected from a letter of the alphabet or a single digit number such as, for example although not limited to, an A-shape, B-shape, S-shape, Z-shape, figure-2, figure-5 and figure-8 which are capable of forming at least two independent airflow/vapour pathways, and a K-shape, M-shape, V-shape, W-shape, X-shape, Y-shape and figure-3 which are capable of forming three or more independent longitudinal airflow/vapour pathways through the vapour chamber when the storage sachet is opened.

In one embodiment the passive evaporation support material is adapted to provide three or more independent longitudinal airflow/vapour pathways. The pathways may be formed as independent conduits through the passive evaporation support material itself or the pathways may be formed by the evaporative means making contact with an internal surface of the vapour chamber. Accordingly, in one embodiment, the passive evaporation support material comprises three or more longitudinal conduits wherein the conduits are formed within the passive evaporation support material or are formed by the passive evaporation support material together with an internal surface of the vapour chamber or a combination thereof when the storage sachet is opened.

The passive evaporation support material may be made from any material that is suitable for absorbing the inhalable liquid and passively releasing it as a vapour. Materials which have wicking properties may be suitable passive evaporation support material for use in the present device. Wicking properties will generally be understood to include the ability of a material to facilitate or enhance the rate of evaporation or vaporisation of a liquid from its surface by distributing the liquid, whether by drawing, spreading, pulling or otherwise, throughout the material from its initial point of contact and/or as it evaporates from an exposed surface area of the material. Accordingly, in one embodiment the passive evaporation support material is a wicking material. In one embodiment the wicking material is a wicking felt or a porous polymeric material. In a preferred embodiment the wicking material is a polypropylene wicking felt.

In one embodiment there is provided an inhaler device for the delivery of an inhalable liquid to a patient according to the first aspect or alternative aspect as herein described, wherein the inhaler body inhaler body comprises:
(1) A base end;
(2) A mouthpiece end comprising a mouthpiece chamber;
(3) An air intake chamber comprising at least one air inlet hole;
(4) An air exit chamber adapted to internally receive an air filtering means within the elongated body and comprising at least one air outlet hole optionally located in the base end; and
(5) an internal shelf to partially divide the elongated body along its longitudinal axis from the base end and terminating at the mouthpiece chamber to form the floor of the air intake chamber and the roof of the air exit chamber;
wherein the storage sachet is located within the air intake chamber.

In one embodiment the internal shelf is planar or non-planar and optionally comprises one or more recessed portions.

In another embodiment the device further comprising a two-way valve abutted to the internal shelf at the mouthpiece end whereupon respiration by a patient through the mouthpiece end:

(a) upon inhalation by the patient, opens the two-way valve between the air intake chamber and the mouthpiece chamber to deliver the evaporated liquid in the form of a vapour to the patient and closes the two-way valve between the air exit chamber and the mouthpiece chamber; and
(b) upon exhalation by the patient, opens the two-way valve between the air exit chamber and the mouthpiece chamber to exhaust the expired air and closes the two-way valve between the air intake chamber and the mouthpiece chamber.

In an alternative embodiment the device further comprising a one-way valve between the air intake chamber and the mouthpiece chamber and/or a one-way valve between the mouthpiece chamber and the air exit chamber.

In one embodiment the air inlet hole further comprises an air intake control means.

In another embodiment the air filtering means comprises activated carbon.

In one embodiment there is provided an inhaler device for the delivery of an inhalable liquid to a patient, said device comprising:
(1) An inhaler body comprising an air intake chamber, an air exit chamber and a mouthpiece chamber;
(2) At least one air inlet opening;
(3) At least one vapour inhalation opening;
(4) At least one air outlet opening; and
(5) A storage sachet comprising a vapour impermeable film or foil for sealingly storing a passive evaporation support material pre-loaded with the inhalable liquid;
   wherein the stored liquid is released from the passive evaporation support material into the inhaler body in the form of a vapour for administration to a patient when the sachet is opened and further wherein the air inlet opening and vapour inhalation opening provide an air/vapour intake pathway through the air intake chamber and mouthpiece chamber upon inhalation by the patient and the vapour inhalation opening and the air outlet opening provide an air/vapour exit pathway through the mouthpiece chamber and air exit chamber upon exhalation by the patient. The storage sachet for use in the device is as described herein and is located within the air intake chamber.

In one embodiment the air intake chamber comprises an air inlet opening, the air exit chamber comprises an air outlet opening and the mouthpiece chamber comprises a vapour inhalation opening.

In one embodiment the inhaler body is an elongated body which will generally adopt the same cross-sectional shape along its length. In one embodiment the cross-sectional shape of the elongated body is selected from circular, semi-circular, elliptical, semi-elliptical, oval, ovoidal, square, rectangular, trapezoidal, triangular and combinations thereof. Shapes having square corners may also be replaced with rounded corners, for example, a rectangle having a square corner replaced by a rounded one may be referred to as a rounded rectangular shape. In one embodiment the cross-sectional shape of the elongated body is selected from cylindrical, rectangular, rounded rectangular, trapezoidal and rounded trapezoidal. The cross-sectional shape of the mouthpiece chamber may be the same or different to the rest of the elongated body. In one embodiment, the mouthpiece chamber is tapered towards the vapour inhalation opening. In a preferred embodiment the cross-sectional shape of the vapour inhalation opening is adapted to fit a conventional aerosol or nebuliser face mask.

In one embodiment the air exit chamber is located within the mouthpiece chamber. In an alternative embodiment, the device comprises an internal shelf to partially divide the inhaler body along its longitudinal axis and terminating at the mouthpiece chamber to form the floor of the air intake chamber and the roof of the air exit chamber.

It will be understood that the use of the terms ‘floor’ and ‘roof’ of the inhaler body, air intake and air exit chambers as used herein are relative terms only and solely as a point of reference with respect to the orientation of the device as envisaged for normal operation. It is envisaged that in normal operation, the device will be oriented so that the storage sachet is position within the air intake chamber so as to provide a volume of air above it into which the vapour can evaporate and further when the inhaler body comprises an air exit chamber that the air intake chamber is positioned above the air exit chamber. However, the device is envisaged to function in alternative orientations.

In one embodiment, the air inlet opening further comprises an air intake control means. In a further embodiment the air intake control means is an adjustable cover located to adjustably cover or close the air inlet opening(s). The air inlet opening(s) may be formed in a number of ways when the adjustable cover is opened, for example, by groove(s) or hole(s) in the air intake chamber which may be exposed to provide an air flow pathway or by groove(s) or hole(s) which may optionally align with groove(s) or hole(s) in the adjustable cover.

When the device is activated for patient use by opening the storage sachet, the adjustable cover may be gradually adjusted from a closed position where it completely covers the air inlet hole(s), to a partially opened or fully opened position to enable the air to flow into the air intake chamber and across the surface(s) of the passive evaporation support material to deliver the vapour to the patient as the patient inhales. In one embodiment the adjustable cover is selected from the group consisting of a rotatable end cap cover located at a base end of the elongated body; a sleeve cover rotatably mounted around the circumference of the elongated body; a slideable cover and a flap cover. The rotatable end cap cover and rotatable sleeve cap cover may be detachably fastened to rotatingly engage with the rest of the elongated body of the device by, for example, a screw thread arrangement or a snap-fit joint arrangement.

The adjustable cover may also advantageously enable the device to be temporarily and/or partially sealed when the adjustable cover is in a closed position to prevent excess vapour escaping through the air inlet hole(s) during intermittent use. Accordingly, in one embodiment the adjustable cover is a rotatable end cap optionally comprising a wad insert. The wad insert may comprise a compressible material and a vapour impermeable film or foil to assist with providing a tight seal when the rotatable end cap is closed. Examples of compressible materials include but are not limited to polymeric foams or sponges such as LDPE. Examples of vapour impermeable films and foils are as described herein.

In another embodiment, the adjustable cover optionally comprises vents to restrict rather than completely prevent the intake of air through the air inlet hole(s) when the adjustable cover is in a closed position.

In use, the air inlet hole(s) may be opened in a number of ways. The adjustable cover may be opened, for example, by popping, pulling, twisting, turning, rotating, unscrewing, sliding, pivoting or flipping the adjustable cover open relative to the elongated body. The air flow pathway may be adjustably controlled by the degree of popping, pulling, twisting, turning, rotating, unscrewing, pivoting or sliding of the adjustable cover relative to the elongated body to provide partially opened or fully opened air inlet opening(s). The adjustable cover may comprise one or more air inlet opening(s) to adjustably align with the air inlet opening(s) in the air intake chamber.

It may also be desirable to filter the exhaled air which contains a proportion of the inhaled vapour in order to reduce the exposure of others in close proximity to the patient during administration. In one embodiment the air exit chamber is adapted to internally receive an air filtering means. In one embodiment, the air exit chamber comprises the air filtering means.

Examples of air filtering means include but are not limited to activated carbon (‘AC’), preferably in granular form. In one embodiment the air filtering means comprises carbon, preferably in granular form. In another embodiment the air filtering means is a cartridge comprising an air filtering substance such as activated carbon (‘AC’), preferably in granular form and/or one or more filters such as optimised filter paper(s). The cartridge may be insertably removable from the air exit chamber or may be integrally formed therein. In one embodiment the cartridge comprising the air filtering substance(s) is insertably removable from the air exit chamber by for example, a sliding guide means in the air exit chamber wall(s) and/or in the internal shelf. In another embodiment, the cartridge comprising the air filtering substance(s) is integrally formed with the air exit chamber wall(s) and/or the internal shelf. In one embodiment the air exit chamber is adapted to internally receive activated carbon granules. In a further embodiment the activated carbon granules are present within the air exit chamber.

It may also be desirable to increase or decrease the size of the air intake chamber relative to the air exit chamber without having to substantially increase the overall size of the device. Accordingly, it is considered that one advantage of the present device is the ability to provide different ratios of air intake chamber size to air exit chamber size depending on design requirements. In one embodiment, the internal shelf is positioned within the elongated body to divide the internal volumes of the air intake chamber to the air exit chamber in a ratio selected from within the range 5:95 to 95:5 or 10:90 to 90:10. In one embodiment, the ratio is selected from the group consisting of 5:95, 10:90, 15:85; 20:80, 25:75, 30:70, 35:65, 40:60, 45:55; 50:50, 55:45, 60:40, 65:35; 70:30, 75:25; 80:20, 85:15, 90:10 and 95:5. Ratios in-between are also contemplated.

In one embodiment the internal shelf is positioned to provide an internal volume of the air intake chamber to an internal volume of the air exit chamber in a ratio selected from the group consisting of 50:50, 55:45, 60:40, 65:35; 70:30, 75:25; 80:20, 85:15, 90:10 and 95:5 and vice versa. In one embodiment the relative size of the air intake chamber to the air exit chamber is >50%. In a further embodiment the internal volume of the air intake chamber to the internal volume of the air exit chamber is in a ratio selected from the group consisting of 55:45, 60:40, 65:35; 70:30 and 75:25. In another embodiment the relative size of the air intake chamber to the air exit chamber is <50%. In a further embodiment the internal volume of the air intake chamber to the internal volume of the air exit chamber is in a ratio selected from the group consisting of 45:55, 40:60, 35:65; 30:70 and 25:75. In one embodiment the ratio is 50:50.

The internal shelf may be planar or non-planar. In one embodiment the internal shelf is planar. However, in order to achieve the desired ratios while still accommodating the passive evaporative support material within the air intake chamber and the air filtering means within the air exit chamber, a non-planer configuration may be preferred. In one embodiment the internal shelf is non-planar. Examples of non-planar configurations may comprise one or more recessed portions or adopt the same or a similar cross-sectional profile as the elongated body, for example a semi-circular cross-section when the elongated body is cylindrical. In one embodiment the internal shelf is planar or non-planar and optionally comprises one or more recessed portions. In another embodiment the internal shelf is non-planar and adopts the same or a similar cross-sectional profile to the elongated body.

In another embodiment, the device comprises a two-way valve abutted to the internal shelf at the mouthpiece end to direct the airflow/vapour pathway through the device whereupon respiration by a patient through the mouthpiece end:

(a) upon inhalation by the patient, opens the two-way valve between the air intake chamber and the mouthpiece chamber to deliver the evaporated liquid in the form of a vapour to the patient and closes the two-way valve between the air exit chamber and the mouthpiece chamber; and
(b) upon exhalation by the patient, opens the two-way valve between the air exit chamber and the mouthpiece chamber to exhaust the expired air and closes the two-way valve between the air intake chamber and the mouthpiece chamber.

In one embodiment the two-way valve abuts up against the internal shelf and is connected thereto. In another embodiment the two-way valve is inserted to abut up against the internal shelf without being connected thereto.

In an alternative embodiment there is optionally provided one or more one-way valves to direct the airflow/vapour pathway through the device. In a further embodiment there is provided a one-way valve between the air intake chamber and the mouthpiece chamber and/or a one-way valve between the mouthpiece chamber and the air exit chamber.

In one embodiment there is provided an inhaler device for the delivery of an inhalable liquid to a patient according to the first aspect or alternative aspect as herein described, wherein the inhaler body comprises a receptacle and a receptacle lid.

In one embodiment the inhaler is a puck-shaped body.

In another embodiment the air inlet opening and the vapour inhalation opening provide an air/vapour pathway through the inhaler body when the receptacle lid is in a opened position.

In a further embodiment the receptacle lid is rotatably openable.

In one embodiment the receptacle lid comprises serrated projections which are adapted to perforate, puncture or pierce open the vapour impermeable film upon rotating the lid open.

In yet another embodiment the storage sachet is formed from a vapour impermeable film and a base portion wherein the base portion is the receptacle and the storage sachet is sealed by sealing a perimeter edge of the base portion with an outer perimeter of the vapour impermeable film.

In yet another embodiment there is provided an inhaler device for the delivery of an inhalable liquid to a patient, said device comprising:
(1) An inhaler body comprising a receptacle and a receptacle lid;
(2) At least one air inlet opening;
(3) At least one vapour inhalation opening; and
(4) A storage sachet comprising a vapour impermeable film or foil for sealingly storing a passive evaporation support material pre-loaded with the inhalable liquid;
   wherein the stored liquid is released from the passive evaporation support material into the inhaler body in the form of a vapour for administration to a patient when the sachet is opened and further wherein the air inlet opening and vapour inhalation opening provide an air/vapour pathway through the inhaler body when the lid is in an opened position.

The storage sachet for use in the device is as described herein. In one embodiment the storage sachet is formed from a vapour impermeable film having a base portion wherein the base portion is formed by the receptacle. The storage sachet may therefore be sealed by sealing the vapour impermeable film to a lip of the receptacle.

In one embodiment the inhaler body is a puck-shaped body. While the inhaler body is described as being ‘puck-shaped’ it will be understood that variations may be accommodated. For example, the receptacle lid may be curved e.g. domed, instead of flat or it may also adopt a different external shape by virtue of, for example, the addition of optional grips to assist the user with opening the lid. The puck-shaped body may also be elongated or truncated with reference to its height although in one embodiment, there is provided a truncated puck-shaped body having a flat top (i.e. top of receptacle lid) and flat bottom (i.e. bottom of receptacle) to provide a slim-line and readily stackable device for pocket-sized storage and portability.

In one embodiment, the air inlet opening and the vapour inhalation opening are formed in the receptacle lid. In a further embodiment, the receptacle lid comprises an integrally formed mouthpiece for delivery of the vapour via the vapour inhalation opening.

In one embodiment the receptacle lid is rotatably openable in relation to the receptacle. When required for use, the air inlet opening(s) and vapour inhalation opening(s) are in an open position to enable the air to flow into the inhaler body and across the surface of the passive evaporation support material to deliver the vapour to the patient as the patient inhales.

The air inlet opening(s) and vapour inhalation opening(s) may be formed in the puck-shaped body in a number of ways to provide an air flow pathway through the inhaler body. For example, the opening(s) may be formed by groove(s) or hole(s) in the receptacle or receptacle lid which may be exposed when the lid is opened or may be formed by groove(s) or hole(s) in the receptacle lid which partially or fully align with corresponding groove(s) or hole(s) in the receptacle when the lid is adjustably opened.

In one embodiment, the air inlet opening and the vapour inhalation opening are formed in the receptacle lid to provide an air flow pathway through the vapour chamber when the lid is opened to deliver the vapour to the user when the user inhales. In another embodiment, the air inlet opening and the vapour inhalation opening are formed in both the receptacle lid and the receptacle to provide an air flow pathway through the device when the lid is adjustably opened to adjustably control the airflow pathway by partially or fully aligning the opening(s) in the lid with the opening(s) in the receptacle to deliver the vapour to the user when the user inhales. In one embodiment, the receptacle lid comprises an integrally formed mouthpiece for delivery of the vapour via the vapour inhalation opening.

The receptacle lid may be opened, for example, by popping, upward pulling, twisting, turning, rotating or unscrewing the lid relative to the receptacle. In one embodiment the receptacle lid is rotatably opened. The receptacle lid may be detachably fastened to sealingly engage with the receptacle by, for example, a screw thread arrangement or a snap-fit joint arrangement. The air flow pathway may be adjustably controlled by the degree of popping, upward pulling, twisting, turning, rotating or unscrewing of the lid relative to the receptacle to provide partially opened or fully opened opening(s). The receptacle lid may also advantageously enable the device to be temporarily sealed by closing the lid to prevent excess vapour escaping through the opening(s) during intermittent use.

The receptacle lid may optionally comprise a wad insert to assist with sealing and resealing the device for storage mode. The wad insert may comprise a compressible material and a vapour impermeable film or foil to assist with providing a tight seal when the receptacle lid is closed. Examples of compressible materials include but are not limited to polymeric foams or sponges such as LDPE. Examples of vapour impermeable films and foils are as described herein.

The devices described herein may be made from various materials. However, suitable material(s) may be selected by considering whether they are chemically inert, stable and impervious with reference to the inhalable liquid to be stored and/or delivered. Material(s) may also be selected based on their suitability for medical device applications such as by reference to whether they meet approved standards for medical-grade human use by a regulatory authority like the FDA.

It is envisaged that the present device will be particularly useful for storing and/or administering halogenated volatile liquids. Accordingly, in one embodiment, the device is made from one or more materials that are compatible with the storage and/or delivery of halogenated volatile liquids to a patient, in particular methoxyflurane for use as an analgesic.

Examples of materials which may be suitable for making the present device include but are not limited to polymers (including homopolymers and heteropolymers i.e. co-polymers), composites (including nanocomposites), metals (including alloys thereof) and combinations thereof. In one embodiment, the device is made from polymers (including homopolymers and heteropolymers i.e. co-polymers), composites (including nanocomposites such as polymers in combination with clay), metals (including aluminium and alloys thereof) and combinations thereof. In a further embodiment, the device is optionally internally lined or coated with one or more material(s) selected from the group consisting polymers (including homopolymers and heteropolymers i.e. co-polymers), composites (including nanocomposites such as polymers in combination with clay), metals (including aluminium, nickel and alloys thereof), oxides (including aluminium oxides, silicon oxides), resins (including epoxyphenolic resins and ionomeric resins such as Surlyn®, trademark of DuPont), lacquers and enamels.

The inhaler body of the device may be formed as a single manufactured part. Embodiments of the device may require additional manufactured parts such as for example an air intake control means, an air filtering means, and the vapour impermeable films and base portion of the storage sachet as described herein. Each manufactured part may be separately formed from the same or a different material. In one embodiment, the separately manufactured parts of the device are independently made from a material selected from the group consisting of a polymeric material, a metal (for example, aluminium, nickel) and a metal alloy (for example, stainless steel).

Polymers are particularly suited to large scale manufacturing of the present device and polymeric films described herein, in particular the inhaler body and the vapour impermeable film and the base portion of the storage sachet, by injection moulding, blow moulding and extrusion processes. They may also be suitable for manufacturing the present device on a smaller scale by 3D printing techniques. Further, polymers may be recycled following disposal of the device.

Examples of polymers for use in making the present device and polymeric films described herein may include but are not limited to the following polymers and combinations (including co-extruded polymers) thereof: polyolefins such as polypropylene (‘PP’), polyethylene (‘PE’) including low density (‘LDPE’), linear low density (‘LLDPE’) and high density polyethylene (‘HDPE’), biaxially orientated polypropylene (‘BOPP’), 4-methylpentene, polymethylpentene, polycyclomethylpentene; polymeric phthalates such as polyethylene naphthalates (‘PEN’), polyethylene terephthalate (‘PET’) (also known as (‘PETE’)), polyethylene terephthalate polyester (‘PETP’), polyethylene isophthalate (‘PEI’), polybutylene terephthalate (‘PBT’), polytrimethylene terephthalate (‘PTT’), polycyclohexylenedimethylene terephthalate (‘PCT’); fluorinated polymers including polymers fluorinated after manufacture (e.g. fluorination post-moulding), fluorinated ethylene-propylene, chlorotrifluoroethylene (‘Kel-F’), polytetrafluoroethylene (‘PTFE’); polyesters including cellulose acetate, polyoxymethylene (‘POM’) and polyesters containing a terephthalate ester group including co-polymers such polyethylene terephthalate glycol co-polyester (‘PETG’), polycyclohexylenedimethylene terephthalate glycol modified (‘PCTG’) and polycyclohexylenedimethylene terephthalate/isophthalic acid (‘PCTA’); nylons including amorphous nylon; polyvinyls including polyvinyl alcohol (‘PVA’) and ethylene vinyl alcohol (‘EVOH’); polysulfones including polyethersulfone (‘PES’); and natural polymers including starch, cellulose and proteins. Suitable polymers may also include polymers with a moisture vapour transmission rate (‘MVTR’, also known as water vapour transmission rate ‘WVTR’) of less than 100 g/m²/24 h, preferably less than 50 g/m²/24 h.

Accordingly, in one embodiment the device is made from one or more polymers wherein the device further comprises an optional internal lining or coating with one or more material(s) selected from the group consisting polymers (including homopolymers and heteropolymers (also known as co-polymers) and combinations thereof including co-extruded polymers), composites (including nanocomposites such as polymers in combination with clay), metals (including aluminium, nickel and alloys thereof), oxides (including aluminium oxides, silicon oxides), spray coatings, resins (including epoxyphenolic resins and ionomeric resins such as Surlyn®, trademark of DuPont), lacquers and enamels.

In one embodiment the polymer is selected from a polyolefin, a polymeric phthalate, a fluorinated polymer, a polyester, a nylon, a polyvinyl, a polysulfone, a natural polymer and combinations, including co-extruded polymers thereof. In one embodiment the polymer has a MVTR of less than 100 g/m²/24 h, preferably less than 50 g/m²/24 h. In one embodiment the polyolefin is selected from the group consisting of PP, PE, LDPE, LLDPE, HDPE, 4-methylpentene, polymethylpentene, polycyclomethylpentene and combinations, including co-extruded polymers thereof such as BOPP. In one embodiment the polymeric phthalate is selected from the group consisting of PEN, PET, PETP, PEI, PBT, PTT, PCT and combinations, including co-extruded polymers, thereof. In one embodiment the fluorinated polymer is selected from Kel-F, PTFE and combinations, including co-extruded polymers thereof. In one embodiment the polyester is selected from the group consisting of cellulose acetate, POM and polyesters containing a terephthalate ester group including PETG, PCTG, PCTA and combinations, including co-extruded polymers, thereof. In one embodiment the nylon is an amorphous nylon. In one embodiment the polyvinyl is selected from PVA, EVOH and combinations, including co-extruded polymers, thereof. In one embodiment the polysulfone is PES. In one embodiment the natural polymer is selected from the group consisting of starch, cellulose, proteins and combinations, including co-extruded polymers, thereof.

In one embodiment the device is made from a single polymer selected from the group consisting of PP, PE, LDPE, LLDPE, HDPE, BOPP, 4-methylpentene, polymethylpentene polycyclomethylpentene, PEN, PET, PETP, PEI, PBT, PTT, PCT, Kel-F, PTFE, cellulose acetate, POM, PETG, PCTG, PCTA, nylon, PVA, EVOH, starch, cellulose, proteins and combinations, including co-extruded polymers, thereof. In another embodiment the device is made from two or more polymers selected from the group consisting of PP, PE, LDPE, LLDPE, HDPE, 4-methylpentene, polymethylpentene polycyclomethylpentene, PEN, PET, PETP, PEI, PBT, PTT, PCT, Kel-F, PTFE, cellulose acetate, POM, PETG, PCTG, PCTA, nylon, PVA, EVOH, starch, cellulose, proteins and combinations, including co-extruded polymers, thereof. In one embodiment, the device is made from a polymer selected from the group consisting of HDPE, PET and combinations thereof. In one embodiment the device comprises PET.

As the inhalable liquid may be self-administered by a patient using the device, the device may optionally comprise a lanyard and a point for attachment thereto for placement around the patient's wrist or neck. Accordingly, in one embodiment the device comprises a lanyard and a point for attachment thereto.

### Example 1

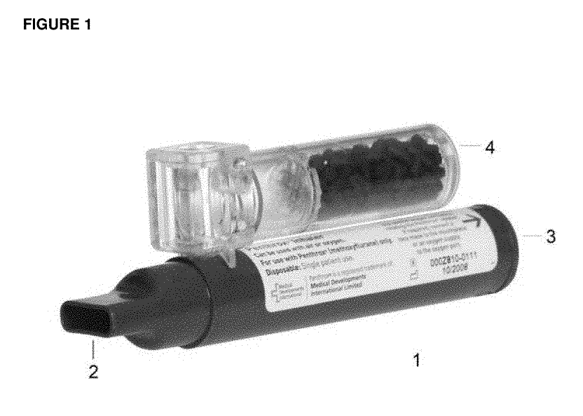

FIG. 1 shows the prior art Green Whistle™ inhaler device (1) (Medical Developments International Limited) which is currently used in Australia for the delivery of Penthrox®/™ (methoxyflurane) as an analgesic (1.5 mL or 3 mL, storage brown glass vial container with screw cap). When required for use, the delivery dose of methoxyflurane is poured into the base end (3) of the device. After the dose is poured into the base end for delivery onto the evaporative means (not shown), the methoxyflurane evaporates so that the patient can self-administer the analgesic by inhaling the air/vapour mix through the mouthpiece (2). Provided that the patient continues to breathe through the mouthpiece, any exhaled air/vapour mix will exit the device via the externally fitted chamber containing activated carbon ‘AC-chamber’ (4).

### Example 2

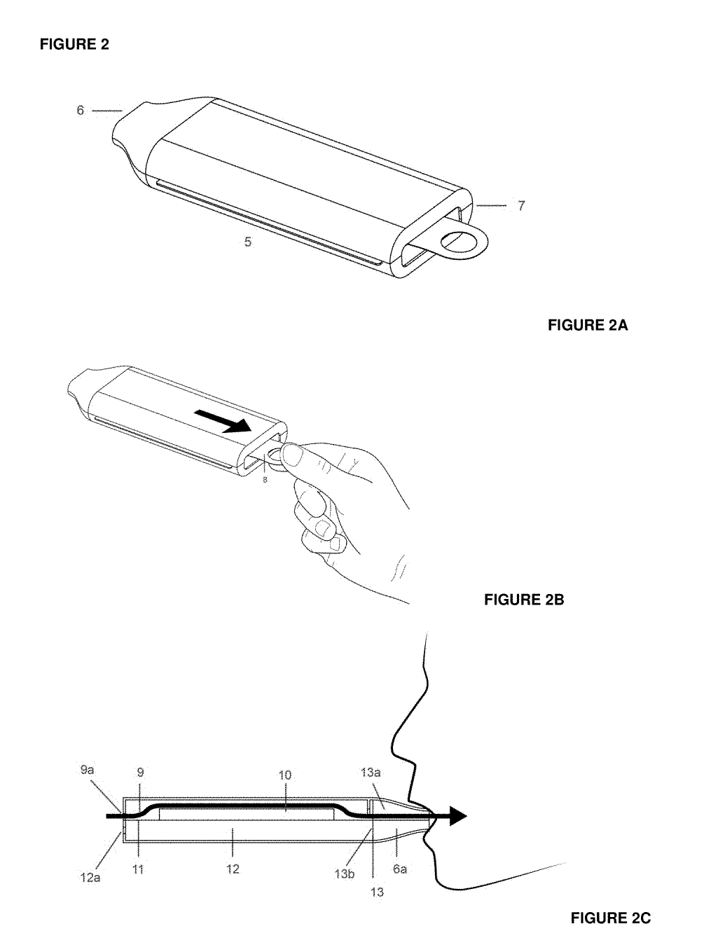

FIG. 2 shows an inhaler device (5) according to an embodiment of the invention. An external view of the device is presented in FIG. 2A showing that the cross-sectional shape of the inhaler body is, with the exception of the tapered mouthpiece (6), a rounded rectangular shape (7). The pull tab (8) of the storage sachet located within the inhaler protrudes from an opening to allow gripping and pulling by the user in the direction of the arrow shown in FIG. 2B to open the storage sachet. The cross-sectional view presented in FIG. 2C shows the inhaler device in use. The storage sachet is positioned within an air intake chamber (9). The air intake chamber comprises an air inlet opening (9a) from which the pull tab protrudes and which enables air to flow through the air intake chamber across the exposed surface(s) of the passive evaporation support material (10) once the storage sachet is opened and into the mouthpiece chamber to deliver the released vapour to the patient when the patient inhales. The direction of the air/vapour pathway upon inhalation is illustrated by the arrows. The storage sachet is positioned on an internal shelf (11). The internal shelf divides the air intake chamber from the air exit chamber (12). The air exit chamber comprises an air outlet opening (12a) to allow the air to exit from the device upon exhalation by the patient. A two-way valve (13) connected to the internal shelf directs the air/vapour flow pathway upon inhalation and exhalation by the patient and further functions to separate the air intake chamber from the mouthpiece chamber (two-way valve portion 13a) and the mouthpiece chamber from the air exit chamber (two-way valve portion 13b). Pulling of the pull tab (8) by the user in the direction indicated (FIG. 2B) opens the storage sachet, thereby releasing the vapour into the air intake chamber for inhalation by the patient (FIG. 2C).

### Example 3

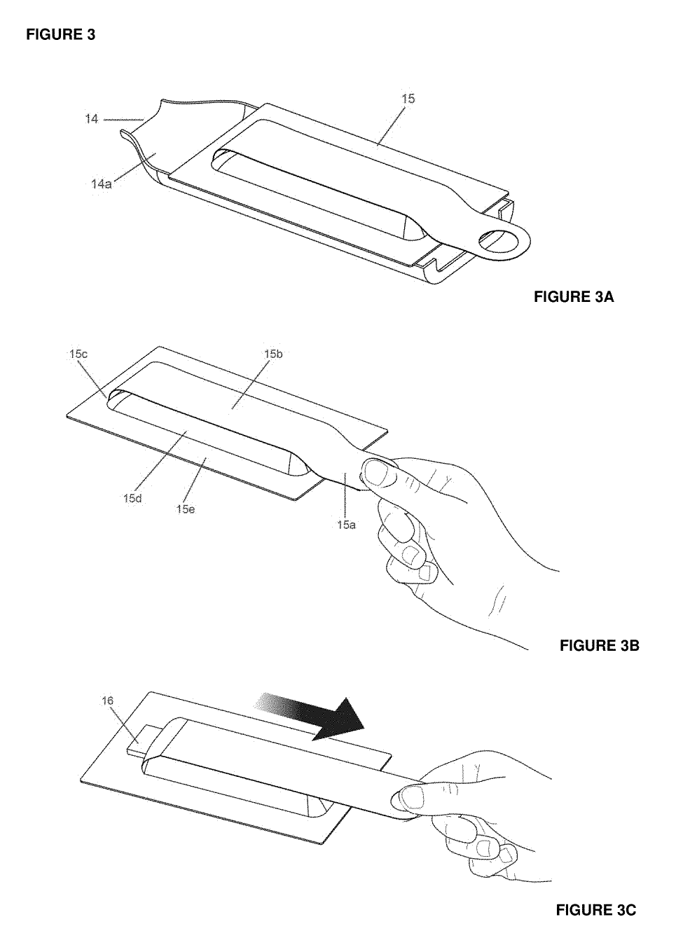

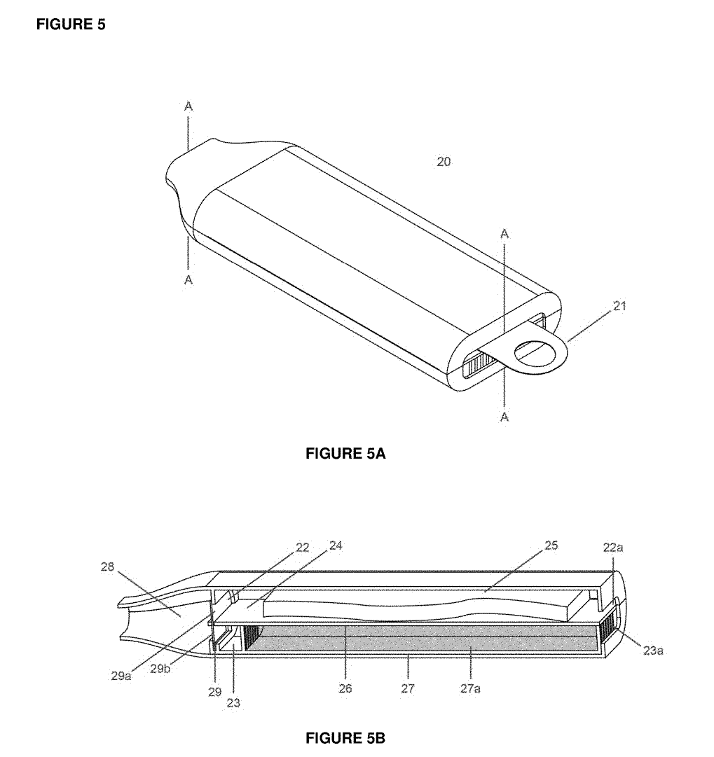

FIG. 3 shows a storage sachet (15) for use in an inhaler device according to an embodiment of the invention. FIG. 3A shows an inhaler device with its upper body portion cut away to show the storage sachet positioned in its lower body portion with the lower body portion comprising the vapour inhalation opening (14) and mouthpiece chamber (14a) visible. The features of the storage sachet are better illustrated by FIG. 5B which shows the pull tab end (15a) of the pull tab (15b) which is connected (15c) to the vapour impermeable membrane portion (15d) of the storage sachet which in turn is sealingly engaged with the rigid or semi-rigid planar base portion (15e). Upon pulling the pull tab in the direction shown by the arrow in FIG. 3C, the vapour impermeable film is removed from the base portion to expose the passive evaporation support means pre-loaded with the inhalable liquid (16) to allow release of the inhalable liquid as a vapour from its surface.

### Example 4

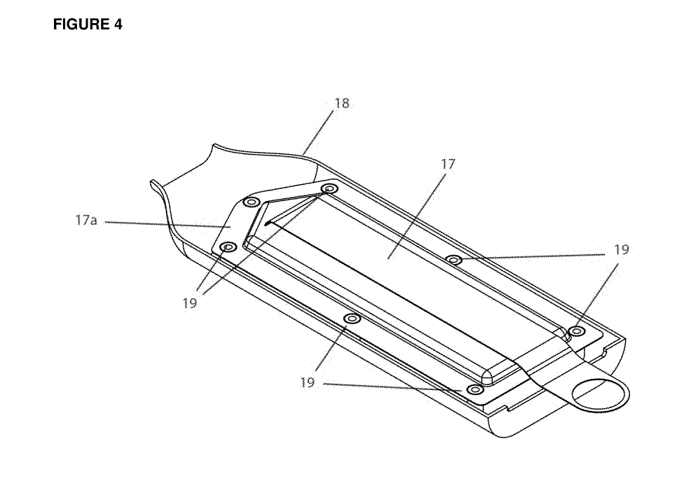

FIG. 4 shows a storage sachet (17) according to an embodiment of the invention fastened by an outer perimeter portion (17a) to a lower portion of an inhaler body (18) by pins (19).

### Example 5

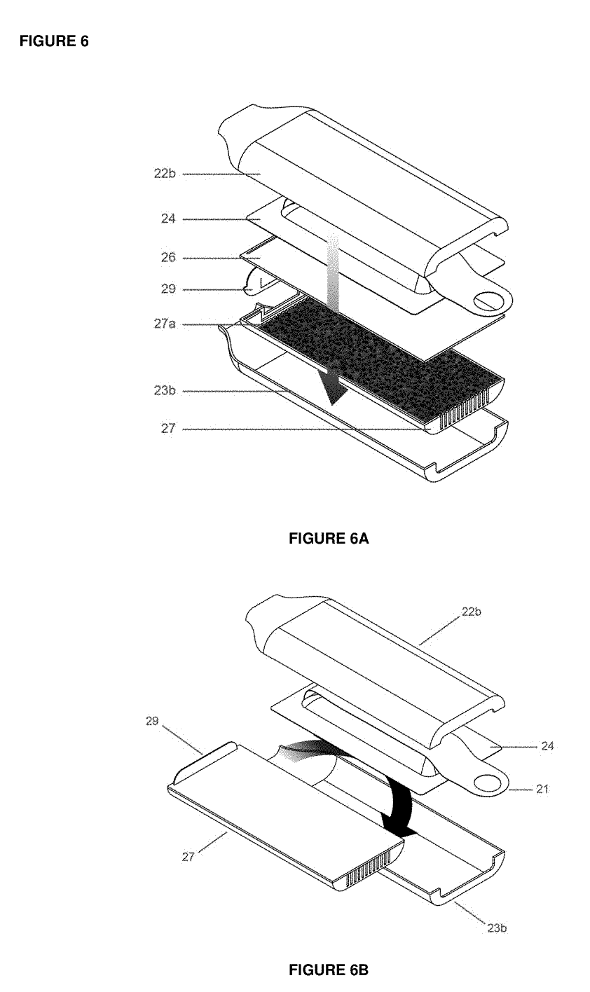

FIG. 5A shows an inhaler device (20) according to an embodiment of the invention comprising a storage sachet. The pull tab (21) of the storage sachet located within the inhaler protrudes from an opening to allow gripping and pulling by the user to open the storage sachet. Cross-sectional view A-A of FIG. 5A is presented in FIG. 5B. The inhaler body of the device comprises an air intake chamber (22) with an air inlet opening (22a) and an air exit chamber (23) with an air exit opening (23a). The storage sachet is located within the air intake chamber and is shown ready for use having been opened to remove the vapour impermeable film with the base portion (24) and passive evaporation support material pre-loaded with the inhalable liquid (25) remaining. An internal shelf (26) separates the air intake chamber from the air exit chamber and also supports the storage sachet. The air exit chamber comprises an air filtering means in the form of an optionally removable cartridge (27) comprising activated charcoal granules (27a). In use and upon inhalation by the user through the mouthpiece chamber (28) the two-way valve (29) which is connected to the internal shelf, opens between the air intake chamber and mouthpiece chamber (two-way valve portion (29a)) and closes between the mouthpiece chamber and the air exit chamber (two-way valve portion (29b)). The air/vapour pathway flows in the direction of the arrows shown, in through the air inlet opening and air intake chamber passing across the surface of the passive evaporation support material and into the mouthpiece chamber to deliver the vapour to the patient. Although not shown, when the user exhales, the two-way valve opens between the mouthpiece chamber and air exit chamber (two-way valve portion (29b)) and closes between the mouthpiece chamber and air intake chamber. The exhaled air/vapour pathway flows through the air exit chamber and is filtered to absorb or remove the exhaled vapour component(s) by the air filtering means in the air exit chamber before exiting the device through the air outlet opening. FIG. 6A shows an exploded view of the inhaler body separated into an upper body portion (22b) and lower body portion (23b) which encase the internal components of the device, some of which are shown including the storage sachet (24), the internal shelf (26), the two-way valve (29) and the cartridge (27) comprising activated charcoal granules (27a). FIG. 6B illustrates one way in which these components may be assembled by the direction of the arrow.

### Example 6

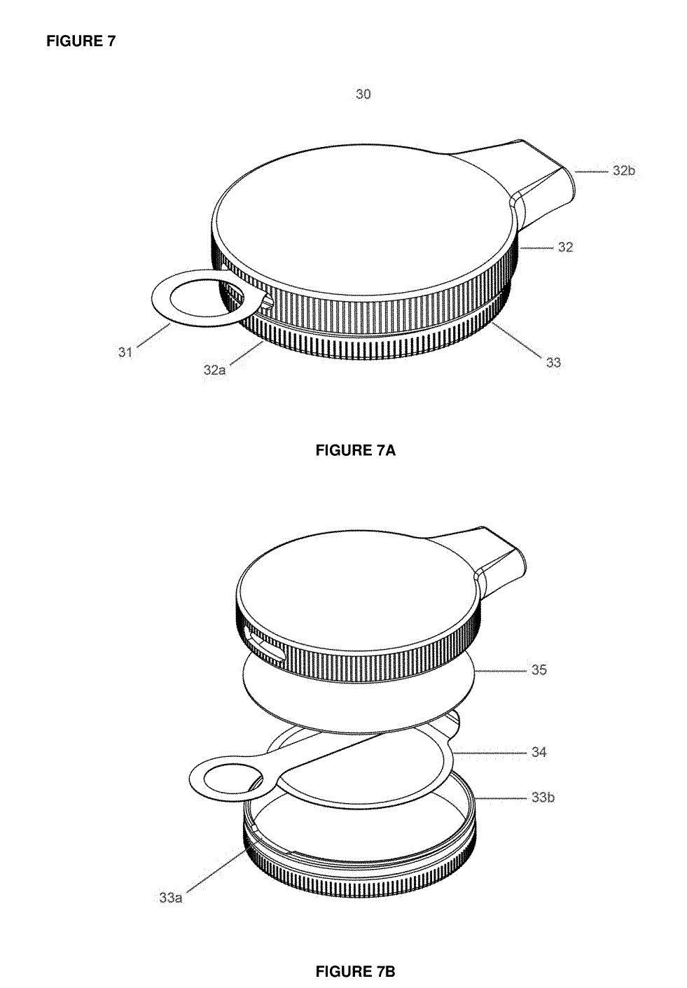

FIG. 7A shows an inhaler device having a puck-shaped body (30) in accordance with an embodiment of the invention. The puck-shaped body comprises a receptacle (33) and a receptacle lid (32), the receptacle lid (32) having an air inlet opening (32a) and an integrally formed and partially tapered mouthpiece with a vapour inhalation opening (32b). An air flow pathway exists between the air inlet opening (32a) and vapour inhalation opening (32b). A pull tab (31) of the storage sachet protrudes from the air inlet opening to allow gripping and pulling by a user. The exploded view presented in FIG. 7B shows the storage sachet (34) having an outer perimeter portion for sandwiching between the lower perimeter edge of the receptacle lid (32) and upper perimeter edge or lip (33b) of the receptacle. Alternatively, the outer perimeter portion of the storage sachet (34) may be fastened to the lip (33b), internal perimeter wall, or floor of the receptacle (33). The passive evaporation support material, pre-loaded with an inhalable liquid, is enclosed within the storage sachet (34). The receptacle lid (32) also comprises a wad insert (35). The upper outer perimeter of the receptacle (33) comprises external circumferential ribs which engage with corresponding internal circumferential grooves (not shown) of the receptacle lid (32) thereby snap-fitting the receptacle lid (32) on to the receptacle (33). The receptacle (33) further defines a notch (33a) which aligns with a lower portion of the air outlet opening (32a).

### Example 7

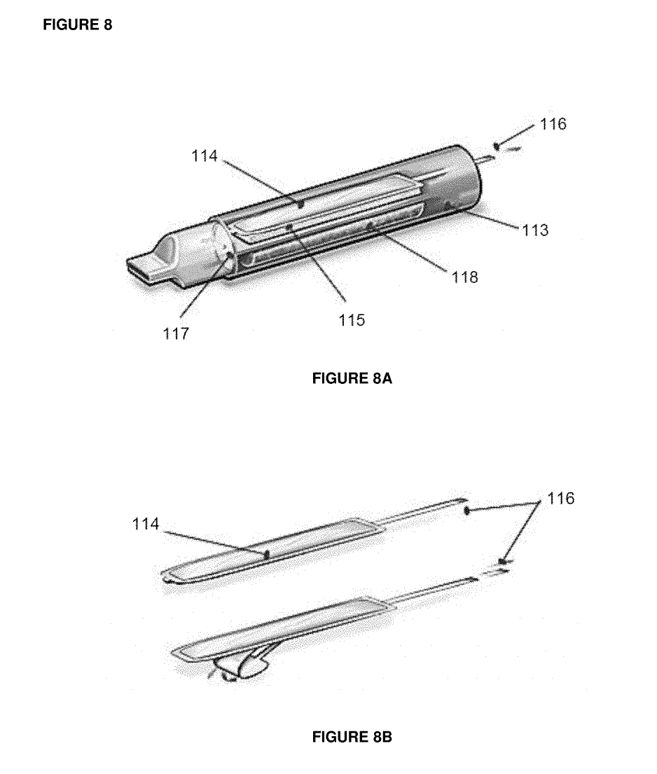

FIG. 8A shows a perspective view of an inhaler device (113) according to an embodiment of the invention. The storage sachet is located within the air intake chamber (114) and positioned above the passive evaporation support material (115). The liquid is released from the storage sachet onto the passive evaporation support material by pulling a tab (116) to open the sachet in the direction of the arrow shown. A two-way valve (117) is provided to control the direction of the air/vapour flow through the air intake chamber and out of the air exit chamber comprising AC (118).

FIG. 8B provides an expanded view of the sachet (114) which is peeled opened in the direction of the arrows shown by pulling the tab (116).

### Example 8

The ability of an inhaler device to delivery methoxyflurane may be tested using a breath simulator system such as a pulmonary waveform generator system.

The delivery of methoxyflurane (% concentration) by the Green Whistle device with the external AC chamber attached and a Prototype device (FIG. 7) according to the invention was measured using a pulmonary waveform generator system. The Prototype device was manufactured as a rapid prototype using a HDPE equivalent material.

The device was tested as follows. The pulmonary waveform generator was set to “Adult” flow conditions (14 breaths per minute) and the concentration logging software and Datex Sensor commenced. For each test, the polypropylene wick was pre-loaded with methoxyflurane (3 mL) to be delivered and the mouthpiece end of the device then inserted into the opening of the pulmonary waveform generator. Concentration logging was commenced for the first minute for the first breaths concentration and then for the next 20 minutes for steady state testing.

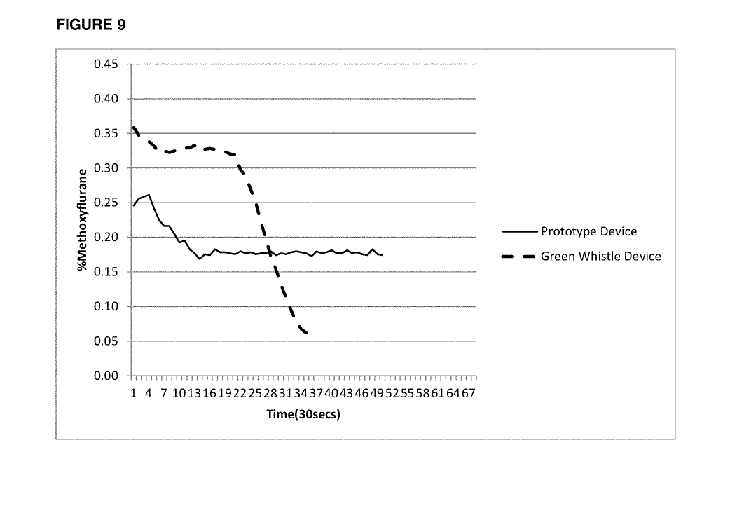

The results are presented in FIG. 9. In both cases, the devices delivered methoxyflurane. While the Green Whistle device initially delivered methoxyflurane at a higher level, the Prototype device was shown to maintain a steady state level over a longer period of time. Accordingly, the Prototype device was shown to deliver a lower concentration of methoxyflurane for a longer duration. In contrast, the Green Whistle device was shown to deliver methoxyflurane at a higher steady state level for a shorter duration initially which was then followed by a rapid reduction below the steady state concentration of methoxyflurane achieved by the Prototype device.

### Example 9

A storage sachet containing a polypropylene wick pre-loaded with methoxyflurane (3 mL) was made from the following materials: an internal/external 20 μm layer of biaxially oriented polypropylene film (‘BOPP’)/adhesive/12 μm layer of Met PET/adhesive/an internal/external 70 μm layer of naturally peelable linear low-density polyethylene.

The stability of the storage sachet may be tested using accelerated storage conditions such as for example in an oven set at 30 C/65% (ICH guideline) and sampled at time periods appropriate for marketing and/or regulatory approval.

Throughout this specification and the claims which follow, unless the context requires otherwise, the word “comprise” and variations thereof such as “comprises” and “comprising”, will be understood to imply the inclusion of a stated integer or step or group of integers or steps but not to the exclusion of any other integer or step or group of integers or steps.

The reference in this specification to any prior publication or information derived from it, or to any matter which is known is not and should not be taken as an acknowledgement or admission or any form of suggestion that prior publication, or information derived from it, or known matter, forms part of the common general knowledge in the field of endeavour to which this specification relates.

1. An inhaler device for the delivery of an inhalable liquid to a patient, said device comprising:
(1) An inhaler body;
(2) At least one air inlet opening;
(3) At least one vapour inhalation opening; and
(4) A storage sachet comprising a vapour impermeable film or foil for sealingly storing a passive evaporation support material pre-loaded with the inhalable liquid;
wherein the stored liquid is released from the storage sachet into the inhaler body in the form of a vapour for administration to a patient when the sachet is opened and further wherein the air inlet opening and vapour inhalation opening provide an air/vapour pathway through the inhaler body.

2. An inhaler device for the delivery of an inhalable liquid to a patient, said device comprising:
(1) An inhaler body comprising a passive evaporation support material for receiving the inhalable liquid;
(2) At least one air inlet opening;
(3) At least one vapour inhalation opening; and
(4) A storage sachet comprising a vapour impermeable film or foil for sealingly storing the inhalable liquid;
wherein the stored liquid is released from the storage sachet into the inhaler body and onto the passive evaporation support material to form a vapour for administration to a patient when the sachet is opened and further wherein the air inlet opening and vapour inhalation opening provide an air/vapour pathway through the inhaler body.

3. The inhaler according to claim 1 wherein the storage sachet comprises a pull tab that protrudes through an opening in the inhaler body whereby it can be gripped and pulled by the user to open the storage sachet.

4. The inhaler according to claim 1 wherein the storage sachet engages with the inhaler body to open the storage sachet wherein the inhaler body comprises a perforating, puncturing or piercing means operable by movement of the device.

5. The inhaler according to claim 1 wherein the storage sachet comprises an outer perimeter portion for fastening to the inhaler body.

6. The inhaler according to claim 1, wherein the inhaler body is adapted to fasten the storage sachet in place.

7. The inhaler according to claim 6 wherein the inhaler body optionally comprises a fastening portion selected from the group consisting of an upper and lower inhaler body portion, an internal rim, a shelf and a floor

8. The inhaler according to claim 6 wherein the storage sachet is fastened in place by sandwiching between an upper portion and a lower portion of the inhaler body which may optionally comprise fastening means or may be fastened by a fastening means to an internal rim, shelf or floor of the inhaler body.

9. (canceled)

10. The inhaler according to claim 1 wherein the inhaler body comprises:
(1) A base end;
(2) A mouthpiece end comprising a mouthpiece chamber;
(3) An air intake chamber comprising at least one air inlet hole;
(4) An air exit chamber adapted to internally receive an air filtering means within the elongated body and comprising at least one air outlet hole optionally located in the base end; and
(5) an internal shelf to partially divide the elongated body along its longitudinal axis from the base end and terminating at the mouthpiece chamber to form the floor of the air intake chamber and the roof of the air exit chamber;
wherein the storage sachet is located within the air intake chamber.

11. The inhaler according to claim 10 wherein the internal shelf is planar or non-planar and optionally comprises one or more recessed portions.

12. The inhaler according to claim 10 further comprising a two-way valve abutted to the internal shelf at the mouthpiece end whereupon respiration by a patient through the mouthpiece end:
(a) upon inhalation by the patient, opens the two-way valve between the air intake chamber and the mouthpiece chamber to deliver the evaporated liquid in the form of a vapour to the patient and closes the two-way valve between the air exit chamber and the mouthpiece chamber; and
(b) upon exhalation by the patient, opens the two-way valve between the air exit chamber and the mouthpiece chamber to exhaust the expired air and closes the two-way valve between the air intake chamber and the mouthpiece chamber.

13. The inhaler according to claim 10 further comprising a one-way valve between the air intake chamber and the mouthpiece chamber and/or a one-way valve between the mouthpiece chamber and the air exit chamber.

14. The inhaler according to claim 10 wherein the air inlet hole further comprises an air intake control means.

15. The inhaler according to claim 10 wherein the air filtering means comprises activated carbon.

16. The inhaler according to claim 1 wherein the inhaler body comprises a receptacle and a receptacle lid.

17. The inhaler according to claim 16 wherein the inhaler is a puck-shaped body.

18. The inhaler according to claim 16 wherein the air inlet opening and the vapour inhalation opening provide an air/vapour pathway through the inhaler body when the lid is in a opened position.

19. The inhaler according to claim 16 wherein the receptacle lid is rotatably openable.

20. The inhaler according to claim 19 wherein the receptacle lid comprises serrated projections which are adapted to perforate, puncture or pierce open the vapour impermeable film or foil upon rotating the lid open.

21. The inhaler according to claim 16 wherein the storage sachet is formed from a vapour impermeable film and a base portion wherein the base portion is the receptacle and the storage sachet is sealed by sealing a perimeter edge of the base portion with an outer perimeter of the vapour impermeable film.

22-58. (canceled)
